(19) United States
(12) Patent Application Publication
(10) Pub. No.: US 2009/0210249 A1
(43) Pub. Date: Aug. 20, 2009

(54) MEDICAL DEVICE

(76) Inventors: Juergen Rasch-Menges, Schwetzingen (DE); Paul Jansen, Mannheim (DE)

Correspondence Address: ROCHE DIAGNOSTICS OPERATIONS INC., 9115 Hague Road, Indianapolis, IN, 46250-0457, US

(21) Appl. No.: 12/370,853

(22) Filed: Feb. 13, 2009

(30) Foreign Application Priority Data
Feb. 16, 2008 (EP) 08002895.4-2201

(51) Int. Cl. G06Q 50/00; A61B 5/145

(52) U.S. Cl. 705/2; 600/365

(57) ABSTRACT

A medical device is provided, comprising a data management unit for providing patient data regarding a patient's health and/or treatment status, a display for displaying the patient data provided by the data management unit, and an actuator configured to cause one or both of activation and deactivation of the device. The patient data is displayed on the display in response to the initiation of activation and/or deactivation of the device.

### CLAIM OF PRIORITY

The present application is a continuation based on and claiming priority to European Patent Application No. 08 002 895.4-2201, filed Feb. 16, 2008, which is hereby incorporated by reference in its entirety.

### TECHNICAL FIELD OF THE INVENTION

The present application relates to a medical device, and more particularly to a device such as a blood glucose meter, a glucose monitoring device, a medical data management device, an infusion device or some other medical device, which collects or processes medical data regarding a patient's health and/or treatment status, and more particularly such a device wherein the data comprises glucose or other analyte concentration measurements taken and/or medication administered.

### BACKGROUND

Medical measuring devices provide a concentration value of an analyte in a body fluid, for example the glucose concentration of blood or interstitial fluid, and store measured concentration values together with the time and date of measuring.

In addition to providing a concentration value, modern medical devices are capable of analyzing a series of measurement values, for example by calculation of an average value, sophisticated statistical evaluation or displaying the trends of concentration values over time. Such an analysis may also be provided by other devices used for the treatment of diabetics, like insulin pens or infusion devices, and is especially valuable for diabetics who need to know as much as possible about the status of their glucose metabolism for optimum treatment of their disease.

Serious long-term secondary effects of diabetes mellitus (for example blindness due to retinopathy) can be prevented only by keeping the blood glucose level within narrow limits at all times by means of accurately dosed administrations of insulin. Optimal adjustment of the dosing of insulin is difficult without repeated checks of the development of glucose concentration over time, i.e. an analysis of a series of measurement values of the patient's glucose concentration.

However, few diabetics use such analysis functions of their devices to the full extent. For many users, accessing analysis results regarding a series of prior measurements is tedious, especially as operating controls of small hand-held devices are necessarily small and therefore difficult to handle, particularly for persons whose manual dexterity is impaired by age or disease. Often, patients are only interested in the result of a current measurement, i.e. a glucose concentration of a fluid sample just taken.

Therefore, suboptimal treatment of diabetics is often detected rather late, sometimes only after a series of long-term secondary effects of diabetes show up. If all users of measurement devices were to use analysis functions of their devices routinely, suboptimal treatment, especially suboptimal adjustment of insulin dosing, could be detected much sooner and harm prevented.

It is therefore an object of the present invention to help diabetics to detect problems of their glucose metabolism, especially suboptimal adjustment of insulin dosing, sooner.

### SUMMARY

This object and others that will be appreciated by a person of ordinary skill in the art have been achieved according to the embodiments of the present invention disclosed herein. In one embodiment, the present invention comprises a medical device comprising a data management unit for providing patient data regarding at least one of a patient's health and treatment status, a display for displaying the patient data provided by the data management unit, and an activator configured to initiate one or both of activation and deactivation of the device, wherein the device is configured to display the patient data on the display upon one or both of the activation or deactivation of the device.

The deactivation of a medical device according to the present invention is caused by a deactivation process which comprises a step in which patient data regarding a patient's health and/or treatment status is displayed. Thereby, patient data, for example an analysis result based on a plurality of measurements, is displayed automatically whenever a user turns the device off. To view patient data a user does not need to enter any complex commands or navigate through a menu. Hence, patient data like analysis results can be provided without any additional effort of a user.

The data management unit of a medical device according to the present invention may be an analytical unit for analyzing measurement values provided by a measuring unit. Such a measuring unit, e.g. for determining an analyte concentration of a body fluid, may be an integral part of the device, i.e. provided in a housing together with the data management unit, or be provided as a separate device communicating wirelessly or via a plug-in connection with the medical device comprising the data management unit and the display. Another possibility is that the data management unit simply stores the time of administrations of medication, e.g. insulin, and provides as patient data information about the last administration or an analysis of several administrations.

The present invention facilitates a user's access to his or her patient data, e.g. analysis results based on a plurality of measurements, for example a series of measurements taken over the last few days. Moreover, patient data are provided in an unobtrusive way, although they are displayed automatically. Furthermore, if a user is for any reason not interested in patient data at the time when the device is turned off, he or she can simply ignore the display during the deactivation process and put the device immediately away, for example into a pocket.

The patient data displayed in response to the initiation of the deactivation process may, for example, be based on the frequency of measurements, and the device may further comprise a warning if the frequency is below a desired value. For example, the number of measurements during a predefined time interval, which might be anything from a few hours up to a number of days or even weeks, can be compared with the number of measurements which should have been taken during that time interval, according to a particular treatment plan or other established schedule. Diabetics often do not measure their blood glucose levels as often as required for optimum treatment of their disease. Sometimes this is thc result of simple laziness, but sometimes an established schedule for measurements might be temporarily insufficient because a patient deviates from daily routine which causes changes in his or her glucose metabolism. Such a need for more frequent measurements can be detected by analyzing past measurement values. The data management unit of the device can be configured to detect such a need for more frequent measurements and calculate a desired value to compare with the frequency of measurements performed. To display a warning whenever the device is turned off, is an unobtrusive way to remind users of the necessity of more frequent measurements.

Alternatively or in addition, the patient data which is displayed in response to the initiation of a deactivation process may indicate a user's metabolic status, show a diagram of previous measurement values, or display an average concentration value or the deviation of the present concentration value from an established average of previous measurements for that time of day.

To avoid unnecessary power consumption, the deactivation process may also be initiated automatically when no user requests have been entered for a preset time. Such a time-out function is commonly used to protect batteries of devices like cell phones or pocket calculators from depletion when a user puts the device away without turning it off. According to one embodiment of the present invention, an analysis result based on a plurality of measurements is displayed on the display in response to such an automatic initiation of the deactivation process as well as in response to an initiation by a user via operating controls.

In one embodiment, the deactivation process can be interrupted via the operating controls. This enables a user to access additional information from the device, if the patient data shown during the deactivation process warrants further attention or simply prodded a user's interest in other analysis results covering other aspects. For example, interruption of the deactivation process may cause the displayed analysis result to freeze or open a navigation menu offering further information.

If the deactivation process is initiated via the operating controls, in one embodiment, continued actuation of the operating controls is required during an initial stage after initiation, which may last for at least half a second, or for at least 0.8 seconds. The operation controls may for example be provided as push buttons that have then to be pressed continuously during the initial stage to prevent abortion of the deactivation process. The requirement of continued actuation during an initial stage prevents accidental deactivation of the device. After the initial stage is completed, the deactivation process takes its course without any need for further action of the user. In other embodiments, the analysis result is displayed during the initial stage and a secondary stage of the deactivation process thereafter.

In yet other embodiments of the present invention, a progress indicator, e. g. a progress bar, is displayed during the deactivation process to indicate progression of the deactivation progress. For example, the progress indicator may indicate progression of the initial stage of the deactivation process. After the initial stage, a progress indicator may also be used during the secondary stage of the deactivation process. The progress indicator may inform a user how much longer the operation controls have to be actuated to prevent abortion of the initial stage or how much longer the analysis result will be shown before the deactivation process terminates and the device turns off.

In yet other embodiments, the analysis result is displayed during the deactivation process for at least two seconds, and can be for at least four seconds. A few seconds are typically enough time to perceive an analysis result displayed as a simple graphic or a few digits. More complex analysis results may require longer display times.

In yet other embodiments, patient data is displayed during the whole duration of the deactivation process to maximize the time a user has to view the analysis result. However, it is also possible that the deactivation process continues for a while after the display is turned off. It is also possible to display a sequence of analysis results during the deactivation process. As the amount of information that can be presented comprehensibly on a tiny display is limited, a sequence of analysis results which are displayed one after the other is an advantageous way to provide a user unobtrusively with complex information or analysis results regarding different aspects of his or her measurement history.

In yet other embodiments of the present invention, the analytical unit is configured to employ different analysis procedures to provide as patient data different analysis results from a given set of concentration values and the times and dates of measurement. In this way analysis results can be provided that cover different aspects of a users glucose metabolism or measurement history. In such embodiments, each time the deactivation process is initiated, an analysis result provided by a different analysis procedure may be displayed. Further, different analysis procedures of the analytical unit may be used alternately to provide the analysis result that is shown during the deactivation process. As diabetics use their measurement devices several times a day, they can thus be provided with different analysis results regarding different aspects of their glucose metabolism or measurement history

Additionally, patient data may also be displayed when the device is activated, i.e. in response to being turned on. Such a device function may be preset by the manufacturer and in certain embodiments this function can be changed by a user who would not want to view analysis results prior to a measurement. In other embodiments, the device function that patient data is displayed in response to the deactivation of the device cannot be changed by a user.

The invention is to be explained in more detail by the following figures and examples.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows an exemplary embodiment of a measuring device according to the present invention.

FIG. 2 shows an example of patient data displayed by the device during the deactivation process.

FIG. 3 shows another example of patient data displayed by the device during the deactivation process.

FIG. 4 shows another example of patient data displayed by the device during the deactivation process.

FIG. 5 shows an example of a problematic analysis result provided by the same analysis procedure as the result shown in FIG. 4.

FIG. 6 shows an example of an alarming analysis result provided by the same analysis procedure as the result shown in FIG. 4.

In order that the present invention may be more readily understood, reference is made to the following detailed descriptions and examples, which are intended to illustrate the present invention, but not limit the scope thereof.

### DETAILED DESCRIPTION OF EMBODIMENTS OF THE PRESENT INVENTION

The following descriptions of the embodiments are merely exemplary in nature and are in no way intended to limit the present invention or its application or uses.

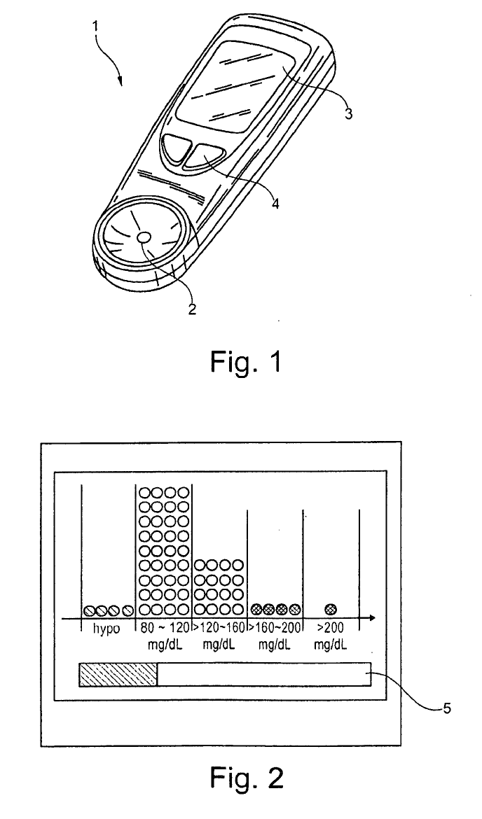

FIG. 1 shows schematically a hand-held measuring device 1 for assaying a sample of a body fluid. The medical device 1 has an integrated lancing facility to puncture a user's finger to collect a sample of blood or interstitial fluid. The device 1 has an opening 2 against which a finger or other body part is pressed for puncturing. A sample collected by puncturing is assayed by a measuring unit (not shown) of the device 1 to determine an analyte concentration of the sample, e. g. its glucose concentration. Measured concentration values can be displayed on a display 3, such as an LCD display.

Instead of having an opening 2 to be pressed against a finger for puncturing, the device 1 may also be configured for use with a separate lancing device and disposable test elements, like strips, onto which a sample is applied and which are then inserted into the device for measuring. The device 1 is typically self-powered, such as by batteries.

Measurement values are stored together with time and date of their measurement in a data storage of the device 1. The device 1 comprises a data management unit provided as an analytical unit (not shown) for analyzing measurement values. The analytical unit may, for example, provide patient data comprising graphics regarding the statistical distribution of measurement values, changes of the analyte concentration over time or perform a statistical analysis of a series of measurement values. Such patient data enable a user to detect, e.g., suboptimal adjustments of insulin dosing at a very early time and thereby helps to prevent serious long-term secondary effects of diabetes mellitus.

In one embodiment of the present invention, the device 1 has operating controls 4 by which a user can operate the device 1 and access its various functions. In the example shown, the operating controls 4 are configured as two push buttons or touch keys. Via these operating elements a user can select items from a navigation menu on the display 3 and thereby view patient data, such as measured concentration values, or analyze results.

To deactivate the device 1, i.e. turn it off, deactivation is initiated via the operating controls 4. The process of deactivation typically comprises an initial stage during which the operating controls 4 are actuated to prevent abortion of the deactivation process and a secondary stage during which no further action of a user is necessary. In the illustrated embodiment, to deactivate the device 1, the left push button is pressed during the initial stage, e.g. for a minimum time of one second. Of course, it is also possible to configure the device 1 in such a way that a different button or several buttons have to be pressed during the initial stage of the deactivation process. Accidental deactivation of the device 1 is prevented by the requirement that the operating controls 4 have to be actuated continuously, such as by a button hold step, during the initial stage of the deactivation process.

In response to the initiation of the deactivation process, according to one embodiment of the present invention patient data, e.g. an analysis result based on a plurality of measurements, is displayed on the display 3. In this way a user can be conveniently provided with patient data regarding his or her metabolic status or measurement history. In other embodiments, patient data is displayed during both the initial stage of deactivation and during the subsequent secondary stage. An example of patient data comprising an analysis result displayed during the deactivation process is shown generally in FIG. 2.

FIG. 2 shows an embodiment of patient data comprising a bar chart in which the length of the bars indicates the percentage of measurements which have yielded a concentration value in a concentration range assigned to each bar. Problematic ranges, i.e. concentration values which are either too low or too high for an optimum glucose metabolism may be highlighted and supplemented by a warning text message. In general, the patient data displayed during the deactivation process may comprise graphic, symbols, numerical digits or text, for example.

In one embodiment, patient data is shown during deactivation for about four seconds. Depending on the complexity of the patient data shown, in other embodiments shorter or longer display times may be appropriate, for example from about two seconds to about ten seconds.

In other embodiments, the deactivation process can be interrupted via the operating controls 4 during the secondary stage while the patient data is displayed. An interruption of the deactivation process may simply prevent the display 4 from turning off for a while, or may open a navigation menu to access other device functions, e.g. abort the deactivation process. A navigation menu displayed in response to an interrupt of the deactivation process may comprise an option to change the set up of the device, i.e. what is displayed when the device 1 is turned on. For example a user may be given the option to change the set up of the device in such a way that patient data like that which is just shown during the interrupted deactivation process is displayed whenever the device 1 is turned on. A menu option to change the set up of the device in such way may be provided only when the deactivation process has been interrupted several times during a predefined time interval, for example a week. A counter may be provided to count how often the deactivation process was interrupted.

According to the embodiment of FIG. 2, the patient data is displayed together with a progress indicator 5 in the form of a progress bar that indicates progression of the deactivation process, such as for the initial phase. In other embodiment, during the secondary stage another progress indicator 5 may inform a user how much time is left for interrupting the secondary stage of the deactivation process, which a user may want to do in order to access additional information regarding the patient data shown.

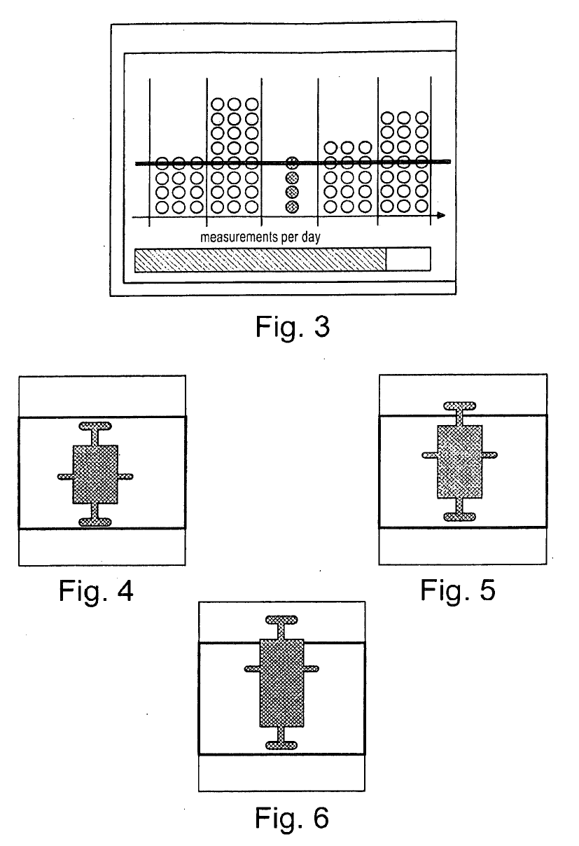

FIG. 3 shows a different example of patient data displayed by display 3 in response to the initiation of the deactivation process. The embodiment of patient data shown in FIG. 3 comprises a bar chart in which the length of the bars corresponds to the number of measurements taken at the respective day. The right-most bar shown in FIG. 3 refers to the current day. The other bars refer to prior days, i.e. to the day before the day of the adjoining bar to their right side. The embodiment of patient data shown in FIG. 3 also comprises a comparison of the frequency of measurements with a desired value. The desired value is indicated by a horizontal bar in FIG. 3. As can be seen, this exemplary bar is superseded only on three days, whereas on two days the bar is barely reached. This indicates that on those two days additional measurements should have been taken. Especially problematic results, for example too few measurements on a given day, can be highlighted by color, arrows or the width of the bar, for example. In other embodiments, it is also possible to highlight holidays and weekends as a person's daily routine on such days is typically different from work days. The glucose metabolism may well reflect that, which warrants special attention to results regarding such days.

FIGS. 4, 5 and 6 show yet other exemplary embodiments of patient data, which can be provided by a different analysis procedure. In FIGS. 4, 5 and 6, a user's metabolic status is indicated by a symbol. In the examples shown, the vertical length of the symbol indicates a range of concentrations which have been measured. Concentrations which have been measured fairly often are covered by the bar, extensions of the bar reach up to the highest or lowest concentration which has been measured during the last seven days. Medically critical concentration values, especially hypo- or hyperglycemic values are indicated by horizontal lines. In FIG. 4, the symbol touches none of the lines which indicates that everything is okay. In FIG. 5, the extension of the bar crosses the upper line which indicates that some problems exist. If the user is aware that a special situation has caused a single unusually high concentration value, the warning implied in FIG. 5 may be ignored. If not, the user might be well advised to change his or her lifestyle or adapted insulin dosing. In FIG. 6, the bar crosses the upper horizontal line which indicates that hyperglycemic values were measured regularly. Therefore, FIG. 6 implies a warning that the user should consult a physician. FIGS. 4 to 6 may be supplemented by appropriate text messages or a background color at reflects the metabolic status, for example the background color may be green, yellow or red depending on the seriousness of the situation.

The device 1 may be configured such that patient data comprising the same analysis procedure is always shown during deactivation. It is also possible to configure the device 1 to show alternately patient data that is provided by different analysis procedures. In this way, the user may be provided with the result of a different analysis procedure whenever the device 1 is turned off. For example the analysis procedures which provide diagrams according to FIGS. 2, 3, and 4 to 6 may be used alternately. It is also possible that the device 1 chooses randomly which of its analysis procedures is used to generate the patient data to be displayed as a response to the initiation of a deactivation process. Another possibility is that during the deactivation process a sequence of patient data is presented, for example first a result according to FIG. 2 and then a result according to FIG. 3, each displayed for a variable, finite number of seconds before the deactivation is completed.

The features disclosed in the above description, the claims and the drawings may be important both individually and in any combination with one another for implementing the invention in its various embodiments.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present invention in detail and by reference to specific embodiments thereof, it will be apparent that modification and variations are possible without departing from the scope of the present invention defined in the appended claims. More specifically, although some aspects of the present invention are identified herein as preferred or particularly advantageous, it is contemplated that the present invention is not necessarily limited to these preferred aspects of the present invention.

What is claimed is:

1. A medical device comprising a data management unit for providing patient data regarding at least one of a patient's health and treatment status, a display for displaying the patient data provided by the data management unit, and an activator configured to initiate one or both of activation and deactivation of the device, wherein the device is configured to display the patient data on the display upon one or both of the activation or deactivation of the device.

2. The device according to claim 1, wherein the activator comprises operating controls on the device, and wherein the deactivation can be interrupted via the operating controls while the patient data is displayed.

3. The device according to claim 1, wherein the patient data is displayed together with a progress indicator that indicates progression of the deactivation.

4. The device according to claim 1, wherein the deactivation comprises an initial stage, during which continued actuation of operating controls is necessary to prevent abortion of the deactivation process, and a subsequent secondary stage, the patient data being displayed during both said stages.

5. The device according to claim 1, wherein the data management unit provides the patient data as an analysis result based on a plurality of one or both of measurement data and medication data.

6. The device according to claim 1, wherein the patient data comprises a comparison of an actual frequency of measurements and a desired value for frequency of measurements.

7. The device according to claim 1, wherein the patient data comprises information relating to a patient's metabolic status.

8. The device according to claim 1, wherein the patient data comprises an analysis result based on a plurality of data relating to one or both of measurements and medication, said data being determined over the course of a previous period of between at least about 24 hours and about five days.

9. The device according to claim 1, wherein the data management unit comprises an integral part of the device.

10. The device according to claim 1, wherein the data management unit is operatively connected to a measuring unit configured for determining an analyte concentration of a sample of a body fluid.

11. The device according to claim 10, wherein the data management unit is connected to the measuring unit via a wireless or wired data communication.

12. The device according to claim 1, wherein the device comprises one of a blood glucose meter, a glucose monitoring device, a medical data management device, and an infusion device.
